(19) United States
(12) Patent Application Publication
(10) Pub. No.: US 2022/0249113 A1
(43) Pub. Date: Aug. 11, 2022

(54) SLIDABLE LOCKING HANDLE DESIGN FOR DEBRIDERS

(71) Applicant: GYRUS ACMI, INC. D/B/A OLYMPUS SURGICAL TECHNOLOGIES AMERICA, Westborough, MA (US)

(72) Inventors: I. Miroslava Avila, Bristol, CT (US); Jeffrey Radziunas, Wallingford, CT (US)

(21) Appl. No.: 17/650,313

(22) Filed: Feb. 8, 2022

Related U.S. Application Data
(60) Provisional application No. 63/147,730, filed on Feb. 9, 2021.

(51) Int. Cl. A61B 17/32

(52) U.S. Cl. CPC A61B 17/32002; A61B 2090/035; A61B 2017/320064

(57) ABSTRACT

A medical device for debridement can include an elongate housing, a shaft extending through the housing and distally therefrom, a drive to manipulate the cutter, and an adjustable handle that can couple with the housing. A lever of the handle can turn about a pivot relative to a handle base to lock and unlock the handle in several orientations useful for debridement.

### CROSS-REFERENCE TO RELATED APPLICATION

This application claims the benefit of priority to U.S. Provisional Patent Application Ser. No. 63/147,730, filed Feb. 9, 2021, the content of which is hereby incorporated by reference in its entirety.

### TECHNICAL FIELD

This document pertains generally, but not by way of limitation, to devices and methods for debridement.

### BACKGROUND

In a medical procedure, such as debridement, a medical device can be used to remove portions of tissue, bone, and/or other objects from a surgical site. The medical device can be a shaver, a debrider, a microdebrider, or other suitable device for debridement. The medical device can have a cutting portion extending from a housing. During the procedure, the housing can be held by a professional and positioned relative to a patient such as to provide precise and careful removal of objects. Modern debriders can incorporate various minimally intrusive cutting mechanisms to shorten recovery time from the procedure. In such minimally intrusive cutting mechanisms, the cutter can be partially enclosed such as to only allow for cutting in a small window of the cutting mechanism. A shaft supplying the cutter can also be curved to enable further precision. Axial rotation of the elongated housing of the medical device can alter the direction of a cutting window and/or the angle of the cutting portion.

### SUMMARY

In an approach to debridement, a medical device can be used such as to abrade and remove portions of tissue or other objects. A medical device can have a cutting mechanism which can be rotatable relative to a housing by manipulation of a nosecone. One example of a medical device can have a housing without a handle and can be gripped by the professional like a pen between a thumb, index finger, and middle finger. Some professionals can prefer pen grip for certain placements and procedures of debridement. Another example of a medical device can have a housing connected to a handle and can be gripped by the professional like a pistol. Some professionals can prefer the pistol grip for certain placements and procedures of debridement and can utilize the ergonomic ease of manipulating the nosecone while the housing is securely anchored by hold of the handle.

Optimal grip of the device can vary among professionals across procedures depending on user hand size and shape, user technique, surgical site location, and other factors. The present inventors have recognized, among other things, that the limited available grip orientations available to a user of a medical device can create the need to alternate between multiple devices or can cause the professional to undertake the procedure without optimal ergonomic position of the device.

Aspect 1 can include or use a medical device for tissue removal, and the medical device can include or use an elongate housing, a shaft extending through the elongate housing and distally therefrom, the shaft containing a cutter configured such as to sever tissue, a drive configured such as to rotate the cutter, reciprocate the cutter, or both; a handle slidably coupled with the elongate housing, and a lever mechanically coupled to a locking mechanism, the locking mechanism configured such as to restrict longitudinal travel of the handle relative to the elongate housing. In Aspect 2, the medical device of Aspect 1 can optionally be configured such that the shaft can be coupled to a vacuum port configured such as to supply remote suction to a lumen of the shaft. In Aspect 3, the medical device of Aspect 1 and/or Aspect 2 can be optionally configured such that the handle can include or use a bore, a lever pivotably connected to the handle, the lever including an eccentric lever cam, and an element movable within the bore configured to be manipulated by the lever cam. In Aspect 4, the medical device of any one or any combination of Aspects 1-3 can optionally be configured such that manipulation from the lever cam can cause the moveable element to protrude from the bore proximally towards the elongate housing. In Aspect 5, the medical device of any one or any combination of Aspects 1-4 can optionally be configured such that manipulation from the lever cam can cause the moveable element to retract into the bore distally from the elongate housing. In Aspect 6, the medical device of any one or any combination of Aspects 1-5 can optionally be configured such that manipulation from the lever cam can cause the moveable element to engage the elongate housing. In Aspect 7, the medical device of any one or any combination of Aspects 1-6 can optionally be configured such that moveable element pressure on the elongate housing can restrict travel of the handle relative to the elongate housing. In Aspect 8, the medical device of any one or any combination of Aspects 1-7 can optionally be configured such that the handle can be slidably coupled to at least one longitudinal groove of the elongate housing. In Aspect 9, the medical device of any one or any combination of Aspects 1-8 can optionally include or use an adjustable handle for use with a medical instrument, and the handle can include or use a handle base, a bore disposed within the handle base, a lever pivotably connected to the handle base, wherein the lever can include or use an eccentric lever cam, and an element movable within the bore configured to be manipulated by the lever cam wherein force on the lever can cause the movable element to apply pressure to a medical instrument, such that the pressure can restrict travel of the handle in the longitudinal groove, such that manipulation from the lever cam can cause the moveable element to articulate between a protruding position wherein the element can extend from the bore proximally towards the medical instrument, and a retracted position wherein the element can be withdrawn into the bore distally from the medical instrument. In Aspect 10, the medical device and/or handle of any one or any combination of Aspects 1-9 can optionally be configured such that the handle can be slidably coupled to the medical instrument. In Aspect 11, the medical device and/or handle of any one or any combination of Aspects 1-10 can optionally be configured such that moveable element pressure on the medical instrument can restrict travel of the handle relative to the medical instrument. In Aspect 12, the medical device and/or handle of any one or any combination of Aspects 1-11 can optionally be configured such that the handle can be slidably coupled to at least one longitudinal groove of the medical instrument. In Aspect 13, the medical device and/or handle of any one or any combination of Aspects 1-12 can optionally be configured such that the lever can be configured to pivotably alternate between an unlocked position wherein the lever can open away from the handle and a locked position wherein the lever can close towards the handle. In Aspect 14, the medical device and/or handle of any one or any combination of Aspects 1-13 can optionally be configured such that travel of the handle relative to the medical instrument can be restricted when the lever is in the locked position. In Aspect 15, the medical device and/or handle of any one or any combination of Aspects 1-14 can optionally be configured such that the lever can be at least partially disposed in a cavity of the handle when the lever is in the locked position. In Aspect 16, the medical device and/or handle of any one or any combination of Aspects 1-15 can optionally be configured such that the at least one longitudinal groove can be a t-shaped slot. In Aspect 17, the medical device and/or handle of any one or any combination of Aspects 1-16 can optionally be configured such that a t-shaped nut can be attached at a protruding end of the moveable element, the t-shaped nut configured such as to apply pressure to the t-shaped slot upon manipulation of the element by the cam. In Aspect 18, the medical device and/or handle of any one or any combination of Aspects 1-17 can optionally be configured such that the moveable element can be biased to travel distally from the medical instrument within the bore. In Aspect 19, the medical device and/or handle of any one or any combination of Aspects 1-18 can optionally be configured such that the moveable element can be biased by a spring within the bore. In Aspect 20, the medical device and/or handle of any one or any combination of Aspects 1-19 can optionally include or use a method of using a medical device with an adjustable handle, and the method can include or use turning a lever about a pivot on a handle body, sliding the handle body relative to a medical instrument, positioning a cutter at a distal end of a shaft, the shaft extending through the elongate housing and distally therefrom, and severing tissue with the cutter. Each of these non-limiting examples can stand on its own, or can be combined in various permutations or combinations with one or more of the other examples.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals can describe similar components in different views. Like numerals having different letter suffixes can represent different instances of similar components. The drawings illustrate generally, by way of example, but not by way of limitation, various embodiments discussed in the present document.

FIG. 1 is a perspective view of a debrider.

FIG. 2A is a side view of a debrider in operation with a pistol grip.

FIG. 2B is a side view of a debrider in operation with a pen grip.

FIG. 3A is a side view of a debrider handle in an unlocked position.

FIG. 3B is a cross-section view of a debrider handle in a locked position.

FIG. 3C is a cross-section view of a debrider handle in an unlocked position.

FIG. 4A is a side view of a debrider in a locked configuration.

FIG. 4B is a side view of a debrider in an intermediate configuration.

FIG. 4C is a side view of a debrider in an unlocked configuration.

FIG. 5A is a cross-section view of a debrider.

FIG. 5B is a perspective view of a debrider handle in a locked position.

FIG. 6A is a side view of an unlocked debrider in a standard configuration.

FIG. 6B is a side view of a partially unlocked debrider in a reverse configuration.

FIG. 6C is a side view of a locked debrider in a reverse configuration.

### DETAILED DESCRIPTION

The present disclosure, in one or more embodiments, relates to devices and methods for surgical debridement. More particularly, the present disclosure relates to an adjustable debrider and methods for versatile and precise debridement. A debridement procedure can be undergone to remove dead, damaged, necrotic, or infected tissue or other objects from a surgical site. A medical device such as a debrider, multidebrider, microdebrider, shaver, morcellator, or other device can be utilized for selective, minimally invasive debridement. Such a medical device may be referred to herein as a debrider. Debridement can be used to remove bone or tissue in ear, laryngeal, paranasal, nasal, uterine, tonsil, adenoid, and skull base surgeries. Debridement can also be used for other beneficial surgical purposes and at other surgical sites.

A debrider can include or use a handpiece, referred to herein as an elongate housing, which can be coupled to a shaft, also referred to herein as a blade. The debrider can be connected to other elements in a debridement system, such as a power supply, suction supply, irrigation line, motor control unit, declog valve, or other elements. The debrider can function to cut, shave, or drill material from a surgical site and can facilitate aspiration of the material from the site and to a collection cannister or other destination. The blade can be removably coupled to the housing and can be sized and shaped according to the specific desired site. Accordingly, a professional can interchangeably couple one of a wide variety of blades to the housing depending on the procedure at hand. Depending on the size and shape of the blade and the surgical site, a professional can alter technique and grip in using the debrider. The housing can also be sized and shaped according to a preferential technique of a professional or to accommodate operation at a specific site. Additional components can be paired to the housing to further modify the device for various uses. One approach to debridement is to use a device having a handle fixed at a predetermined position on the housing. A problem with that approach, however, is that professionals vary greatly in hand size, technique, and other factors. As such, a fixed handle can prevent certain professionals from achieving an ideal hold of the device during the procedure. The stationary handle can provide an uncomfortable or awkward grip to some professionals or can prevent a professional from modifying their hold and technique to accommodate to particular procedures or surgical sites. The present devices and techniques can help avoid such problems because the device is easily and quickly adjustable to several configurations.

The present disclosure relates to, among other things, a device and method enabling debridement at multiple surgical sites. The device can be configured to be held by a professional during debridement using any of several common techniques. Additionally, the device can be configured to be held by an adjustable handle. Alternatively or additionally, the device can be configured to be held by the device housing. The device, when held by the housing, can be configured to be securely held and easily rotated due to presence of an upper portion of the handle. The device can be configured to allow for various secure holding positions of the device with a dominant hand and ergonomic rotation of a nosecone with a non-dominant hand. Further, the device can be configured to allow for multiple points of connection of the handle along the length of the housing.

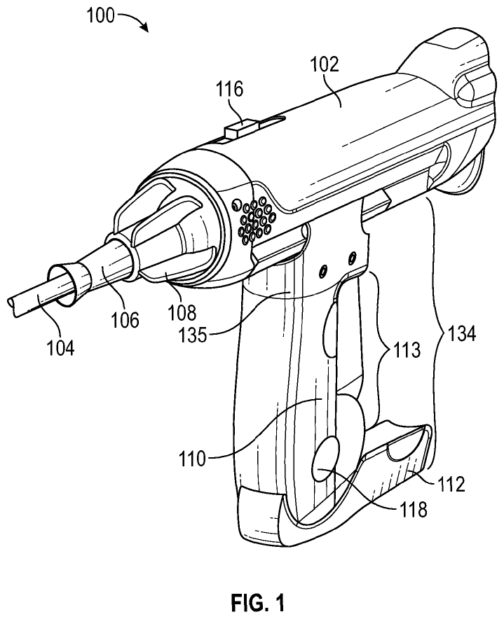

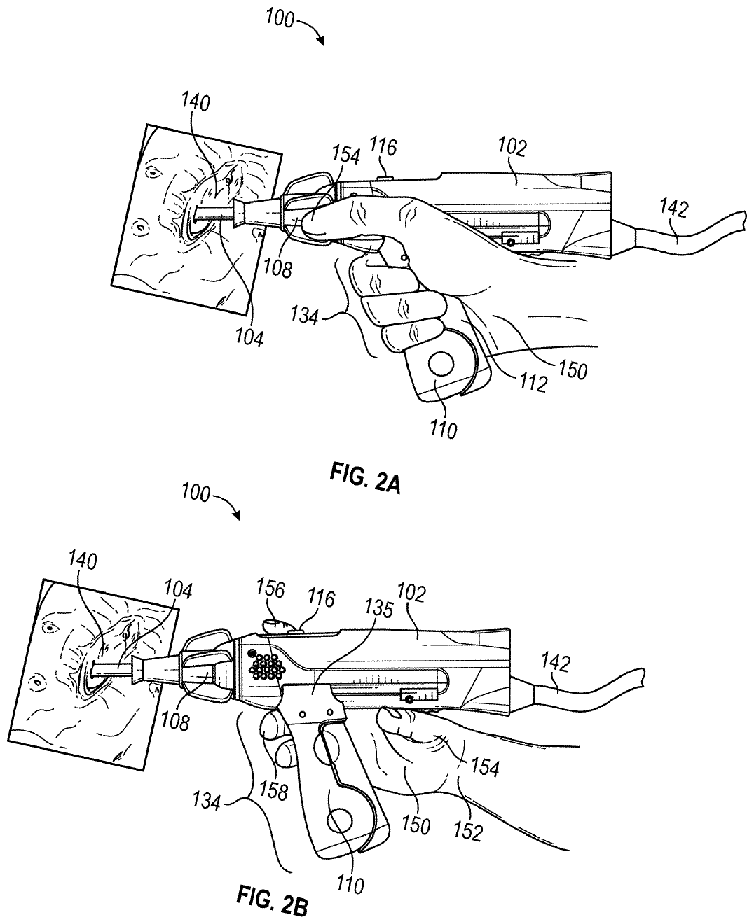

FIG. 1 shows a perspective view of an example of a debrider. A debrider 100 can include or use an elongate housing 102, a blade 106, a cutter 104, a nosecone 108, and an actuator 116. The housing 102 can be elongate and can be substantially cylindrically shaped. The housing 102 can also be substantially elliptically, cylindrically shaped. Diameters of housings 102 shaped as such can be within a range of about 0.25 inches to about 2 inches. In some examples, the housing diameters can be within the range of about 0.875 inches to about 1.25 inches. The housing 102 can be reusable, such as can be washable or autoclavable. Alternatively, the housing 102 can be disposable, its inner contents configurable to a new housing. The debrider 100 can also include or use a handle 134 coupled to the housing 102. The handle 134 can be not intended for removal from the housing 102 once it has been coupled. The handle 134 can be unable to be removed from the housing 102 without excessive force or breakage once it has been coupled. The handle 134 can be sized and shaped such as to interface with the housing 102 by a one-way snap feature, the one way snap feature enabling pairing of the handle 134 with the housing 102 but preventing removal therefrom. The handle 134 can include or use a handle base 110, an upper portion 135 and a lever 112 coupled to the base 110 about a pivot point 118. The blade 106 can be attached or coupled to the housing 102 and extend therefrom. The blade 106 can extend at least partially through the housing 102. The blade 106 can include or use a cutting mechanism, referred to herein as cutter 104. The blade 106 can be curved at a variety of different angles to allow for precision in placement of the cutter 104 during debridement. The cutter 104 can be a reciprocating or oscillating pair of cutting members, a rotating cutting member, or both. Alternatively or additionally, the cutter 104 can be a rotating burr. The cutter 104 can also be an electrode cutter. An illustrative example of an electrode cutter is described in U.S. patent application Ser. No. 15/936,678, filed on Mar. 27, 2018, and entitled Electrode Blade for Shavers, which is incorporated by reference herein in its entirety, including for its teaching of a medical device having an electrode blade, which can be used in combination with the debridement device described in the present disclosure. The cutter 104 can also include or use other mechanisms suitable for debridement blades. The cutter can include a cutter window such as to focus the cutting action in a particular lateral direction relative to the blade 106. The housing can include or use a blade drive mechanism configured to drive the cutter 104. The blade drive mechanism can be an electric motor, a solenoid assembly, or other appropriate mechanism to move the cutter 104. For example, the cutter 104 can be motorized. The blade drive mechanism can be activated by an actuator 116. The actuator 116 can be a switch, a knob, a button, or other mechanism for actuation of the cutter 104. The actuator 116 can be located on the housing 102, such as located on the top of the housing as depicted in FIG. 1. The actuator 116 can be located at other locations on the housing, or alternatively or additionally can be located at a remote location, such as on a footswitch. The blade 106 can include or use a nosecone 108 at or near a blade connection point at a distal end of the housing 102. Turning the nosecone 108 about a medial axis of the housing 102 can cause rotation of the blade 106. Where the cutter 104 of the blade 106 has a cutter window, turning of the nosecone 108 can alter the lateral direction of the cutter window. Further, where the blade 106 is curved at an angle, turning of the nosecone 108 can alter the lateral direction in which the angled blade 106 extends. The debrider 100 can be itself a component of a larger debridement system. For example, the debrider can be tethered to a source of suction, irrigation, or both via one or more connection lines 142 (see FIGS. 2A and 2B). Accordingly, the debrider 100 can provide aspiration and removal of tissue and other objects as they are shaved, cut, or drilled. For example, the debrider 100 can contain a port configured to supply remote suction and/or irrigation to a lumen of the blade or shaft 106. Aspiration can be a helpful feature of a debrider 100 with respect to cutting performance.

FIG. 2A shows a side view of an example of a debrider in operation with a pistol grip. A professional can hold or grasp the debrider 100 by a handle 134 including or using the handle base 110 and the lever 112 coupled to the base 110 about the pivot point 118. Generally, such a hold can be referred to as “pistol grip”. FIG. 2A shows one way a professional can use the debrider 100 in pistol grip. A professional can hold the debrider 102 solely by the handle 134 or can also hold the debrider holding the handle 134 and the housing 102. In one example of a hold depicted in FIG. 2A, a professional can securely grab the handle 134 between a thumb 154 and fingers, the handle 134 being held in an arch of a dominant hand 150. While distinguished herein as a dominant hand 150 and a non-dominant hand, it is understood that either hand can be interchangeably used. Other techniques of holding the handle 134 can be used. Pistol grip can provide a professional with enhanced power and stability in using the debrider 100 to service a surgical site 140. Pistol grip can also enable ergonomic rotation of the nosecone 108 relative to the housing 102 using the non-dominant hand to allow for secure, anchored counterforce applied by the dominant hand 150. Using the nosecone 108 (rather than rotating the housing 102) to rotate the cutter 104 can be beneficial for certain procedures as the housing 102 is generally tethered to the one or more connection lines 142. Pistol grip can further be advantageous for certain techniques and procedures depending on the professional's facility, hand size, and other factors. It can also be used to accommodate various individual patient anatomies and pathologies.

FIG. 2B shows a side view of an example of a debrider in operation with a pen grip. A professional can hold or grasp the debrider 100 by the housing for certain procedures and techniques. Generally, such a hold can be referred to as “pen grip”. FIG. 2B shows one way a professional can use the debrider 100 in pen grip. The housing 102 can rest at or near the dominant hand 150 between a thumb 154 and an index finger 156. The housing 102 can also rest at or near a middle finger 158, as shown. The professional can securely grip the housing between the thumb 154 and index finger 156 and further anchor the hold by gripping the upper portion 135 of the handle 134 with the middle finger 158. Other fingers, such as a ring finger or a pinky finger can also be used to grip the upper portion 135 of the handle 110. Pen grip can enable a professional to have enhanced control and placement of the cutter 104 relative to the surgical site 140. For instance, pen grip with upper portion 135 of the handle 110 as an anchor can enable secure, ergonomic rotation of the debrider 100 without excess strain on a wrist 152. Pen grip can further be advantageous for certain techniques and procedures depending on the professional's facility, hand size, and other factors. It can also be used to accommodate various individual patient anatomies and pathologies. For pen grip, ideal longitudinal placement of the upper portion 135 of the handle 134 relative to the housing 102 can vary based on the size of the professional's hand 150, length of the middle finger 158, technique, and other factors. Further, ideal longitudinal placement of the upper portion 135 of the handle 134 for pen grip can be different than the ideal placement of the same for pistol grip, even for the same professional. Alternatively or additionally, the debrider 100 can be held in pen grip without using the middle finger 158 anchored at the upper portion 135 of the handle 134. For such a use of pen grip, the professional can slide the adjustable handle 134 at a non-impeding longitudinal position, such as can slide the handle 134 to a proximal longitudinal position relative to the housing.

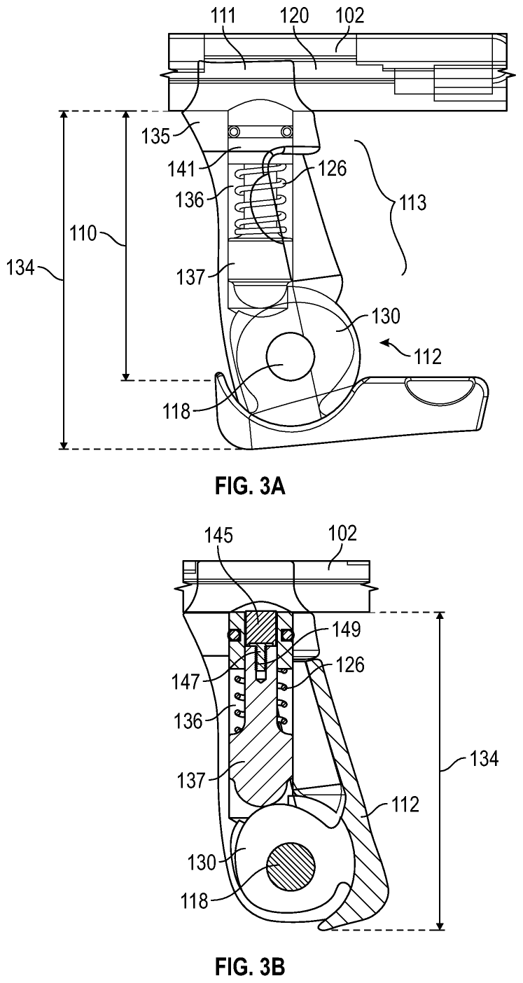

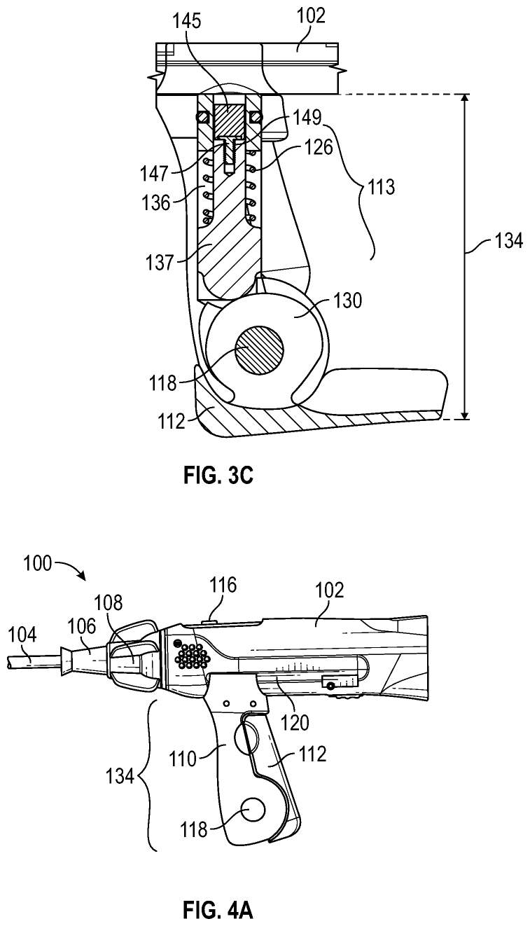

FIG. 3A shows a side view of an example of a debrider handle in an unlocked position. The handle base 110 is shown in phantom in FIG. 3A in order to depict the other components therein. The debrider 100 can include or use mechanisms to allow for variable longitudinal placement of the handle 134 relative to the housing 102. Variable longitudinal placement of the handle 134 can be available in predefined increments, such as by incremental indentations or protrusions on the housing 102. Alternatively, variable longitudinal placement of the handle can be available continuously along the housing 102. In some examples, the debrider 100 can include or use one or more longitudinal grooves of the housing 120. Typically, the debrider 100 can have a pair of longitudinal grooves 120 running parallel on opposing side walls of the housing 102. The upper portion 135 of the handle 134 can include or use one or more bars 111 sized and shaped to ride the one or more longitudinal grooves 120. Where the bars 111 of the upper portion 135 ride the grooves 120, the handle 134 can be slidingly paired to the housing 102. As such, the handle 134 can slide longitudinally along a length of the grooves 120. While the connection is depicted and described herein as having bars 111 riding grooves 120, other equivalent connections enabling longitudinal sliding motion of the handle 112 relative to the housing 102 are commonly known by persons skilled in the art and can be used herewith. For instance, alternatively or additionally the grooves 120 can be on the handle 134 and the bars 111 can be on the housing 102.

The handle 134 can include or use the handle base 110 and the lever 112 connected to the handle base 110 about the pivot 118. The pivot 118 can be a pin, rod, or other substantially cylindrical portion extending through the handle base 110 and the lever 112. The pivot 118 can also be a physical, rotatable connection between the body of the lever 112 and the handle base 110. The pivot 118 can also be one or more projections on the truncate stub 110 which can mate with one or more apertures on the lever 112. The pivot 118 can also be one or more projections on the lever 112 which can mate with one or more apertures on the handle base 110. The handle base 110 can include or use a notch in which to receive a tongue, or cam 130 of the lever 112 at or near the pivot 118 as depicted in FIG. 3A. Alternatively, the lever 112 can contain a notch or dual cam 130 in which to receive a tongue of the handle base 110 at or near the pivot 118. Other appropriate configurations of attachment between the handle base 110 and the lever 112 about the pivot 118 are commonly known by those skilled in the art and can be used herewith. The cam 130 can be substantially elliptically shaped, as depicted in FIG. 3A. Alternatively, the cam 130 can be substantially circularly shaped. The cam 130 can be also be shaped in other ways appropriate to provide cam-action to a follower 137.

The handle base 110 can include or use a bore 136 containing a moveable element. The moveable element can be a follower 137, as depicted in FIG. 3A. The follower 137 can be biased within the bore distally from the housing 102 such as by a spring 126. The spring 126 can bias the follower 137 such as to apply constant pressure of the follower 137 on the cam 130. This constant pressure can cause the follower 137 to remain constantly tangent to the shape of the cam 130. The follower 137 can be at least partially contained in a bushing 141 such as to direct motion of the follower 137 and/or to prevent excess friction within the handle 134 during cam-action. The spring 126 can be fixed to the bushing 141. The cam 130 can be oriented eccentrically about the pivot 118. The cam 130 can be oriented eccentrically such as to convert rotational motion from the lever 112 to linear motion of the follower 137 within the bore 136. Turning the lever 112 radially about the pivot 118 can cause the cam 130 to manipulate the follower 137 to move proximally towards the housing. Additionally, the handle base 110 can include or use a handle cavity or recess 113 which can be sized and shaped to receive the lever 112 when in a locked position.

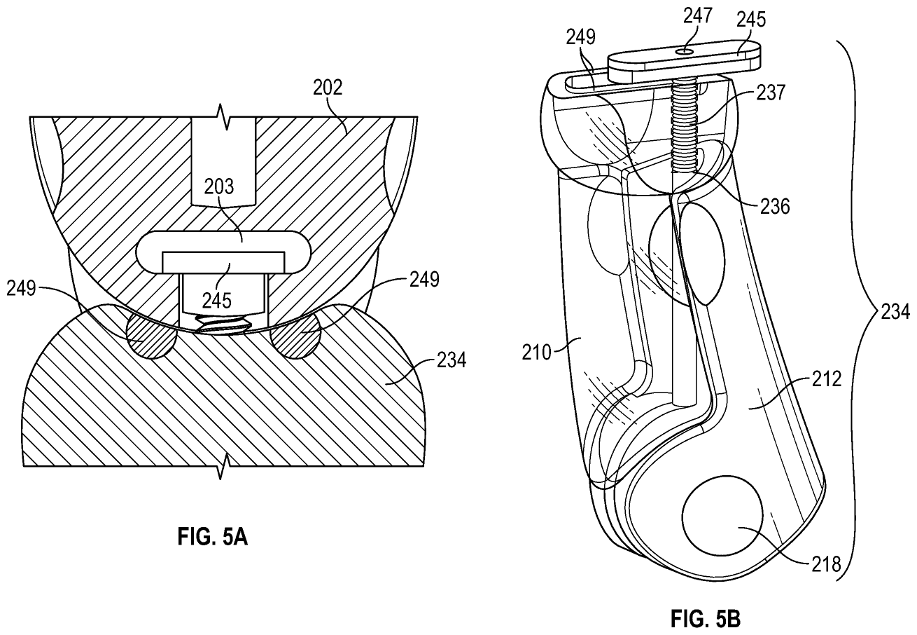

FIG. 3B shows a cross-section view of an example of a debrider handle in the locked position. The cavity or recess 113 can be at least partially enclosed by the lever 112 when in the locked position. The lever 112 can be c-shaped, u-shaped, v-shaped, or can be otherwise shaped such as to mate with the recess 113. Alternatively, the lever 112 can be sized and shaped such as to enclose the housing 102 where the housing 102 does not have a housing recess 113. The lever 112 can be at least partially disposed in the recess 113 of the handle base 110 when in the locked position. Alternatively, the lever 112 can be fully disposed in the recess 113 of the handle base 110 when in the locked position, or can be closed substantially parallel to the base 110 undisposed. The follower 137 can include or use a stopper 145 attached to the opposing end of the follower 137 to that which receives the cam-action. The stopper 145 can be fixed to the follower 137 by a fastener 147 into a threaded hole 149 of the follower. The stopper 145 can alternatively or additionally be attached to the follower 137 using an adhesive. Other appropriate methods of attachment may be used to attach the stopper 145. The stopper 145 can be made of rubber, silicon, plastic, metal, or other material suitable for use in a medical device. The stopper 145 can include or use knurling or other similar surface finish to increase friction during engagement with the housing 102. When the lever 112 is in the locked position, the cam 130 can manipulate the follower 137 to move towards the house 102 against the bias of the spring 136, thus moving the stopper 145 to engage the housing 102. Alternatively or additionally, the housing 102 can include or use a longitudinal channel or slot on its underside (Such as slot 203, depicted below in FIG. 5A). The bushing 141 can protrude into the channel or slot of the housing 102 and can be oriented such as to guide the follower 137 and/or the stopper 145 to engage an inferior surface of the housing 102 inside the channel or slot.

FIG. 3C shows a cross-section view of an example of a debrider handle in an unlocked position. The lever 112 can turn about the pivot 118 radially away from the handle base 110 to the unlocked position. Turning the lever 112 to the unlocked position can cause the cam 130 to release pressure from the follower 137, allowing the follower 137 to move within the bore 136 distally from the housing 102 towards the bias provided by the spring 126. The release of cam 130 pressure on the follower 137 can cause the stopper 145 to disengage from the inferior surface of the housing 102.

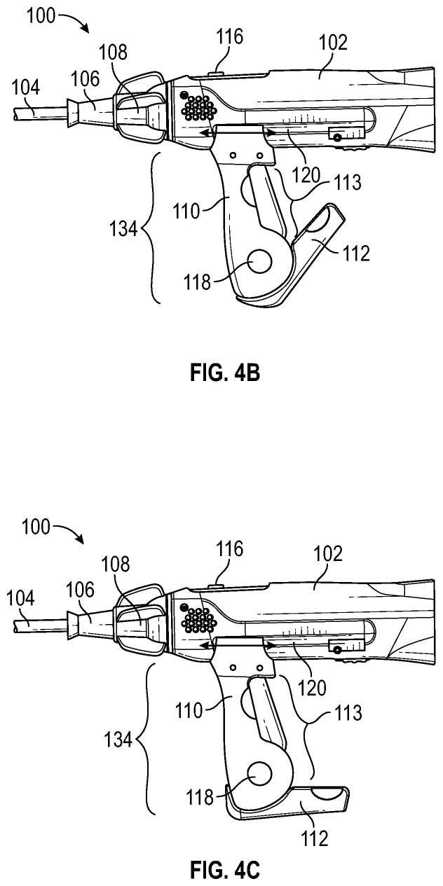

FIGS. 4A-4C show side views of an example of the debrider with varying handle positions. FIG. 4A shows a side view of an example of a debrider in a locked configuration. The handle 134 can extend distally from the housing 102 at a length within a range of about 0.2 inches to about 6 inches. In some examples, the handle 134 can extend at a length within the range of about 2 inches to about 3.5 inches. The handle 134 can, at or near its inferior end, have a girth within a range of about 0.5 inches to about 2.5 inches while in the locked position. In some examples, the handle 134 can, at or near its inferior end, have a girth within a range of about 1 inches to about 1.5 inches while in the locked position. The handle 134, or any of its components, can be formed of polyether ether ketone (PEEK) or other materials suitable for medical use. When the handle is in the locked position, the follower 137 and/or the stopper 145 can be manipulated by the cam 130 to engage an inferior surface of the housing 102. Such engagement can cause the handle 134 to grab or pinch the housing 102, such as grab or pinch the housing 134 between the one or more longitudinal grooves 120 and an inferior surface of the housing 102. Alternatively or additionally, the handle being in the locked position can cause the handle 134 to grow or spread within a slot or channel of the housing 102. The handle 134 being in the locked position, causing the handle 134 to secure the housing, can restrict longitudinal travel of the handle 134 relative to the housing 102.

FIG. 4B shows a side view of an example of a debrider in an intermediate configuration. When the lever 112 is turned radially away from the handle base 110, the handle 134 can release the housing allowing the handle to slide longitudinally relative to the housing 102. The follower 137 can be vertically displaced within the bore 136 relative to an angle at which the lever 112 is turned away from the handle base 110. In one example, the size and shape of the cam 130 can cause the follower 137 to be vertically displaced within the bore 136 linearly relative to an angle at which the lever 112 is turned away from the handle base 110. In another example, the size and shape of the cam 130 can cause the follower 137 to be perpendicularly displaced within the bore 136 harmonically relative to an angle at which the lever 112 is turned away from the handle base 110. For instance, when the lever 112 is turned radially away from the handle base 110 at about 5 degrees, the follower 137 can have essentially no perpendicular displacement. However, when the lever 112 is turned radially away from the handle base 110 at about 6 degrees, the follower 137 can begin to have some perpendicular movement, such as perpendicular movement downward from a bias of the spring 126. Further, when the lever 112 is turned radially away from the handle base 110 from a range of about 20 degrees to about 90 degrees, the follower 137 can have perpendicular movement in a harmonic rise, such as can have significant movement downward from a bias of the spring 126. When the lever 112 is turned radially away from the handle base 110 from a range of about 90 degrees to about 100 degrees, the follower 137 can have little to no perpendicular movement. Such a harmonic relationship of the angular position of the lever 112 to the perpendicular movement of the follower 137 can create a self-locking mechanism. The self-locking mechanism can prevent handle 134 from releasing the housing, enabling longitudinal sliding of the handle 134 relative to the housing 102, where the level is accidentally partially turned. Other harmonic relationships of the angular position of the lever 112 to the perpendicular movement of the follower 137 are known by those skilled in the art and can be used herewith.

FIG. 4C shows a side view of an example of a debrider in an unlocked configuration. The lever 112 can be turned radially away from the handle base 110 to a maximum angle. When the lever 112 is turned radially away from the handle base 110 at the maximum angle, the handle 134 can fully release the housing allowing the handle to freely slide longitudinally relative to the housing 102. Such a maximum angle of the lever 112 can be within a range of about 10 degrees to about 180 degrees. In some examples, such a maximum angle of the lever 112 can be within a range of about 90 degrees to about 120 degrees. In some examples, such a maximum angle can be within a range of about 90 degrees to about 110 degrees.

In operation and use, a professional can provide or obtain the medical device 100 for use in debridement. The professional can turn the lever 112 about the pivot 118 on the handle body 110. The professional can turn the lever 112 radial away from the handle body 110 to move the handle 134 to an unlocked position. With the handle 134 in the unlocked position, the professional can modify the longitudinal placement of the handle 134 relative to the housing 102. The professional can choose a longitudinal position such as to accommodate to their hand size, technique, or the surgical site 140 at hand. In one example, the professional can substantially hold the debrider 100 by the handle 134 to use a pistol grip. For pistol grip, the professional can place the handle 134 relative to the housing 102 such as to create a counterbalance of the device. In another example, the professional can substantially hold the debrider 100 by the housing 102 to use pen grip. For pen grip, the professional can place the handle 134 relative to the housing 102 such as to provide an anchor of the upper portion 135 of the handle 134 to be utilized by the middle finger 158. Alternatively, for pen grip, the professional can place the handle 134 relative to the housing 102 such as towards a proximal end of the housing to prevent the handle from impeding the procedure. The professional can turn the lever 112 completely towards the handle base 110 to place the handle to lock the handle 134 at the desired longitudinal location relative to the housing 102. The professional can quickly and conveniently modify the longitudinal location of the handle 134 during a procedure to accommodate to desired techniques throughout operation. The professional can position the cutter 104 relative to a surgical site 140, and sever, cut, shave, drill, and/or remove tissue or other objects therefrom.

FIGS. 5A and 5B show another example of a debrider handle. Handle 234 is similar to handle 134, but contains a rod 237 as the moveable element within a bore 236. As depicted in FIG. 5A, a housing 202 can have a longitudinal channel or slot 203 on its underside. The slot 203 can be a t-shaped slot. The slot 203 can also be triangular shaped, or can be otherwise shaped for coupling in a similar manner. The handle can contain a clamp 245 sized and shaped to be slidably connected to and mated with the slot 203. The clamp 245 can be, for instance, t-shaped. The handle 234 can also include one or more rails 249. The one or more rails 249 of the handle can be formed of rubber, silicon, plastic, metal, or other material suitable for use in a medical device. The rails 149 can include or use knurling or other surface finish such as to increase friction during engagement with the housing 202.

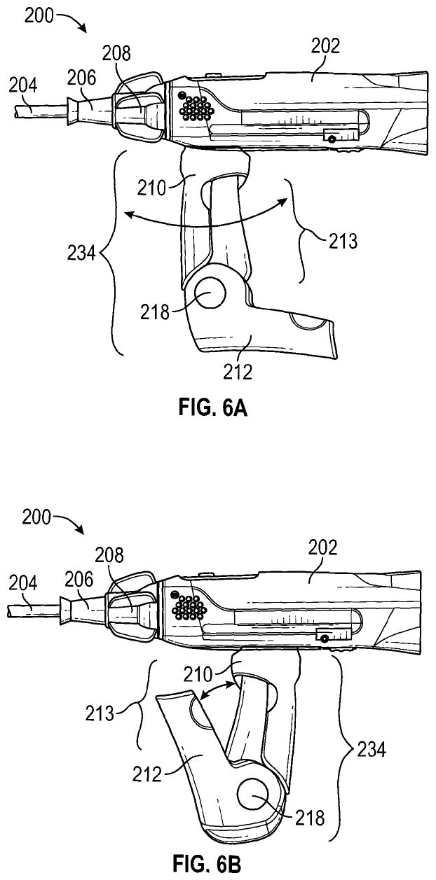

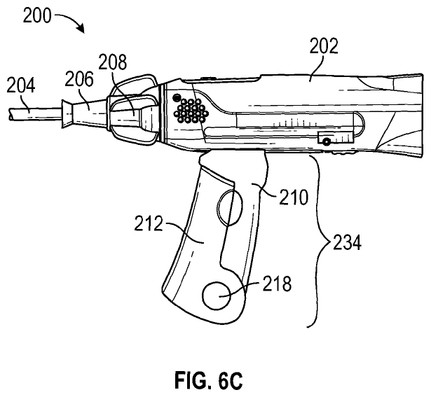

As shown in FIG. 5B, an example of the handle 234 can include or use a handle base 210, a lever 212 connected to the handle base 210 about a pivot 218, a moveable element, such as the rod 237, the clamp 245, and the one or more rails 249. The handle base 210 is shown in phantom in FIG. 5B in order to depict the other components therein. The clamp 245 can be attached to the rod 237 at a superior end of the rod 237. In one example, the rod 237 can be threaded and can be attached to a threaded hole 247 of the clamp 245. Similar to other embodiments described herein, the lever 212 can form an eccentric cam which can manipulate a moveable element, or rod 237, relative to radial turning of the lever 212. The rod 237 can be connected to the cam such as to create a crank-action. Turning the lever 212 radially towards the handle base can pull the rod 237 distally away from the housing. Pulling the rod 237 can cause the clamp 245 to engage with the slot 203. Engagement of the clamp 245 with the slot 203 can pull the handle 234 towards the housing 202 and create pressure from the rails 249 towards an inferior surface of the housing 202. Opposing pressure from the clamp 245 and the rails 249 can cause the handle to secure the housing 202 and can restrict longitudinal movement of the handle 234 relative to the housing 202. Turning the lever 212 radially away from the handle base 210 can push the rod 237 proximally towards the housing 202. Pushing the rod 237 can cause the clamp 245 to release the slot. Relief of opposing pressure from the clamp 245 and the rails 249 can cause the handle 234 to release the housing 202 and can enable longitudinal movement of the handle 234 relative to the housing 202. As depicted in FIGS. 6A-6C, some embodiments of the handle 234 can be rotated axially 360 degrees relative to the housing 202 of the debrider 200 while in an unlocked configuration. Axial rotation of the handle 234, such as axial rotation where the handle 234 is curved or angled, can provide more ergonomic holds to accommodate a variety of professionals in a variety of procedures. When the handle 234 is in an unlocked position, as depicted in FIG. 6A, the rod 245 can provide sufficient clearance between the handle 234 and the housing 202 to allow for rotation of the handle. FIG. 6B depicts the handle 234 in a reverse position, the lever 212 being turned radially to reach the locked position (shown in FIG. 6C).

The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein. In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

The above description is intended to be illustrative, and not restrictive. For example, the above-described examples (or one or more aspects thereof) can be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A medical device for tissue removal, the medical device comprising:
an elongate housing;
a shaft extending through the elongate housing and distally therefrom, the shaft containing a cutter configured to sever tissue;
a drive configured to rotate the cutter, reciprocate the cutter, or both;
a handle slidably coupled with the elongate housing; and
a lever mechanically coupled to a locking mechanism, the locking mechanism configured to restrict longitudinal travel of the handle relative to the elongate housing.

2. The medical device of claim 1, wherein the shaft is coupled to a vacuum port configured to supply remote suction to a lumen of the shaft.

3. The medical device of claim 1 wherein the handle comprises:
a bore;
a lever pivotably connected to the handle, the lever including an eccentric lever cam; and
an element movable within the bore configured to be manipulated by the lever cam.

4. The medical device of claim 3, wherein manipulation from the lever cam causes the moveable element to protrude from the bore proximally towards the elongate housing.

5. The medical device of claim 3, wherein manipulation from the lever cam causes the moveable element to retract into the bore distally from the elongate housing.

6. The medical device of claim 3, wherein manipulation from the lever cam causes the moveable element to engage the elongate housing.

7. The medical device of claim 6, wherein moveable element pressure on the elongate housing restricts travel of the handle relative to the elongate housing.

8. The medical device of claim 1, wherein the handle is slidably coupled to at least one longitudinal groove of the elongate housing.

9. An adjustable handle for use with a medical instrument, the handle comprising:
a handle base;
a bore disposed within the handle base;
a lever pivotably connected to the handle base, the lever including an eccentric lever cam; and
an element movable within the bore configured to be manipulated by the lever cam wherein force on the lever causes the movable element to apply pressure to a medical instrument, the pressure restricting travel of the handle in the longitudinal groove;
wherein manipulation from the lever cam causes the moveable element to articulate between:
a protruding position wherein the element extends from the bore proximally towards the medical instrument; and
a retracted position wherein the element is withdrawn into the bore distally from the medical instrument.

10. The handle of claim 9, wherein the handle is slidably coupled to the medical instrument.

11. The handle of claim 10, wherein moveable element pressure on the medical instrument restricts travel of the handle relative to the medical instrument.

12. The handle of claim 11, wherein the handle is slidably coupled to at least one longitudinal groove of the medical instrument.

13. The handle of claim 11, wherein the lever is configured to pivotably alternate between:
an unlocked position wherein the lever opens away from the handle; and
a locked position wherein the lever closes towards the handle.

14. The handle of claim 13, wherein travel of the handle relative to the medical instrument is restricted when the lever is in the locked position.

15. The handle of claim 14, wherein the lever is at least partially disposed in a cavity of the handle when the lever is in the locked position.

16. The handle of claim 12, wherein the at least one longitudinal groove is a t-shaped slot.

17. The handle of claim 16, wherein a t-shaped nut is attached at a protruding end of the moveable element, the t-shaped nut configured to apply pressure to the t-shaped slot upon manipulation of the element by the cam.

18. The handle of claim 12, wherein the moveable element is biased to travel distally from the medical instrument within the bore.

19. The handle of claim 18, wherein the moveable element is biased by a spring within the bore.

20. A method of using a medical device with an adjustable handle, the method comprising:
turning a lever about a pivot on a handle body;
sliding the handle body relative to a medical instrument;
positioning a cutter at a distal end of a shaft, the shaft extending through the elongate housing and distally therefrom; and
severing tissue with the cutter.
